(19) United States
(12) Patent Application Publication
(10) Pub. No.: US 2017/0071570 A1
(43) Pub. Date: Mar. 16, 2017

(54) ULTRASONIC DIAGNOSTIC DEVICE AND SYSTEM

(71) Applicant: GE MEDICAL SYSTEMS GLOBAL TECHNOLOGY COMPANY, LLC, Waukesha, WI (US)

(72) Inventor: Tadashi JUMATSU, Tokyo (JP)

(21) Appl. No.: 15/126,104

(22) PCT Filed: Mar. 25, 2015
(86) PCT No.: PCT/US2015/022523

§ 371 Date: Sep. 14, 2016

(30) Foreign Application Priority Data
Mar. 26, 2014 (JP) 2014-063956

(51) Int. Cl. A61B 8/00; A61B 6/00; A61B 8/08

(52) U.S. Cl. CPC A61B 8/4477; A61B 8/4405; A61B 8/565; A61B 8/4438; A61B 8/465; A61B 8/54; A61B 8/5215; A61B 6/467

(57) ABSTRACT

An ultrasonic diagnostic device is provided to improve the work flow of an operator. The ultrasonic diagnostic device includes a plurality of ultrasonic probes, and a selection unit 82 that performs a selecting function according to a program so as to select the most frequently used ultrasonic probe based on information on the frequency of use of the ultrasonic probes, the information being specified by information on a use situation of the ultrasonic diagnostic device. The information on the frequency of use is created by the information creation unit 81. The information on the frequency of use may be created for each subject, each operator, or each test type.

### FIELD OF THE INVENTION

The present invention relates to ultrasonic diagnostic device that automatically selects an ultrasonic probe and sets a preset, and a system including the ultrasonic diagnostic device.

### BACKGROUND ART

Kinds of ultrasonic probes are connected to ultrasonic diagnostic device depending on, for example, the type of test. If kinds of ultrasonic probes are connected to the ultrasonic diagnostic device, an operator selects an ultrasonic probe to be used, on an operation unit (For example, see Patent Literature 1: Japanese Patent Publication No. 2005-110739 (page 3)).

Moreover, presets set for the used ultrasonic diagnostic device vary depending on the type of test. For example, depending on the tested part, different measurement buttons or body patterns are displayed on the touch panel of the operation unit, different parameters are provided for transmission and reception of ultrasonic waves, or different functions are allocated to the hard keys of the operation unit.

### SUMMARY OF INVENTION

However, an ultrasonic probe is selected or a preset is set for each type of test, leading to a complicated operation for an operator. Thus, a work flow during a test needs to be improved.

An invention devised to solve the problem according to one aspect is ultrasonic diagnostic device including: a plurality of ultrasonic probes; and a processor that performs a selecting function according to a program so as to select the most frequently used ultrasonic probe based on information on the frequency of use of the ultrasonic probes, the information being specified by information on the use situation of the ultrasonic diagnostic device.

An invention according to another aspect is ultrasonic diagnostic device including a processor that performs, according to a program, a locating function that locates the ultrasonic diagnostic device and a setting function that sets the use condition of the ultrasonic diagnostic device according to a position specified by the locating function.

According to the invention of one aspect, the program is performed by the processor so as to select the most frequently used ultrasonic probe. Thus, an operator does not need to select the ultrasonic probe, improving the work flow of the operator.

According to the invention of another aspect, the use condition is set according to a position specified by the locating function. Thus, a test type may be specified according to the position of the ultrasonic diagnostic device located in, for example, a hospital. In this case, the use condition is set according to a position specified by the locating function and thus the operator does not need to set the use condition according to the test type. This can improve the work flow of the operator.

### BRIEF DESCRIPTION OF DRAWINGS

FIG. 1 is a block diagram showing a schematic configuration of ultrasonic diagnostic device according to a first embodiment.

FIG. 2 is a plan view of the ultrasonic diagnostic device with an illustrated operation unit.

FIG. 3 is a diagram showing a display on the operation unit with displayed probe selection buttons.

FIG. 4 is a block diagram showing some of the functions of a control unit according to the first embodiment.

FIG. 5 is an explanatory drawing showing an example of information on the frequency of use.

FIG. 6 is a block diagram showing a schematic configuration of ultrasonic diagnostic device according to a second embodiment.

FIG. 7 is a diagram showing a schematic configuration of a system that includes ultrasonic diagnostic device and a server connected to the ultrasonic diagnostic device via a network.

FIG. 8 is a block diagram showing some of the functions of a control unit according to the second embodiment.

FIG. 9 is a diagram showing wireless communication terminals provided in examination rooms.

FIG. 10 shows information on the kinds of ultrasonic probes used in the examination rooms.

FIG. 11 is a block diagram showing some of the functions of the control unit according to a second modification of the second embodiment.

FIG. 12 is a conceptual diagram of preset information.

FIG. 13 is a diagram showing another example of the schematic configuration of the system including the ultrasonic diagnostic device and the server connected to the ultrasonic diagnostic device via the network.

### DETAILED DESCRIPTION

Embodiments of the present invention will be described below with reference to the accompanying drawings.

### First Embodiment

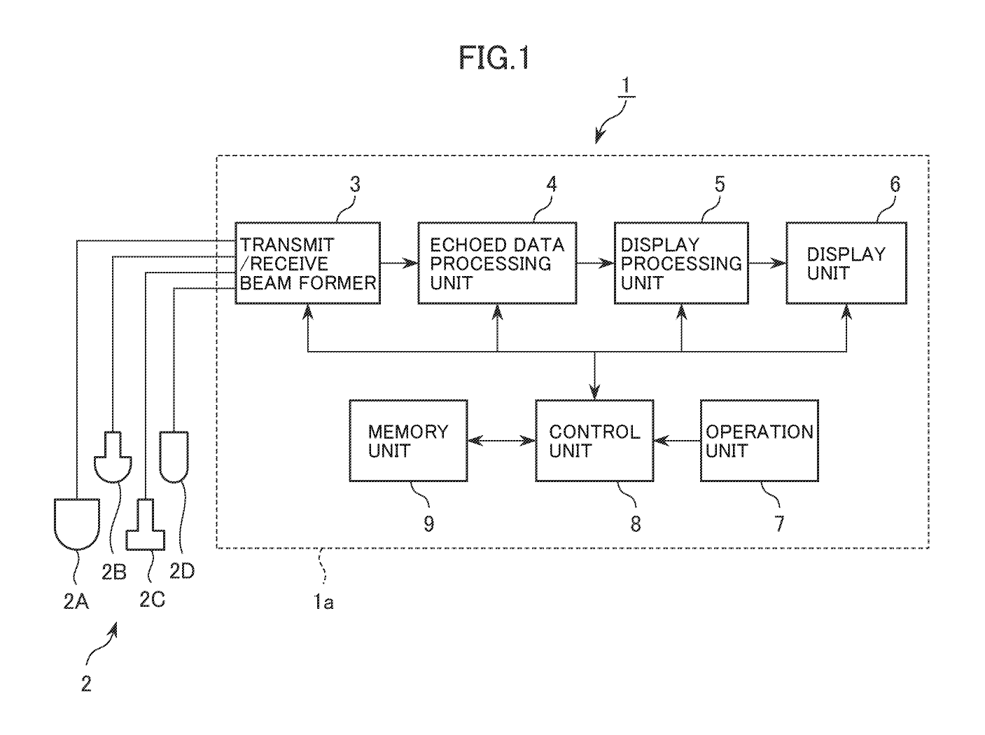

A first embodiment will be described below. Ultrasonic diagnostic device 1 in FIG. 1 includes an ultrasonic probe 2, a transmit/receive beam former 3, an echoed data processing unit 4, a display processing unit 5, a display unit 6, an operation unit 7, a control unit 8, and a memory unit 9. The ultrasonic probe 2 is connected to an ultrasonic diagnostic device body 1a. The ultrasonic diagnostic device body 1a includes the transmit/receive beam former 3, the echoed data processing unit 4, the display processing unit 5, the display unit 6, the operation unit 7, the control unit 8, and the memory unit 9.

The ultrasonic probe 2 is an embodiment of the ultrasonic probe of the present invention. The ultrasonic probe 2 transmits and receives ultrasonic waves to and from a biological tissue of a subject. In this example, four ultrasonic probes 2A, 2B, 2C, and 2D are physically connected to the ultrasonic diagnostic device body 1a via connectors (not shown).

In response to a control signal from the control unit 8, the transmit/receive beam former 3 drives the ultrasonic probe 2 to transmit ultrasonic waves with a predetermined transmission parameter. Moreover, the transmit/receive beam former 3 performs signal processing, e.g., phasing addition on an ultrasonic echo signal.

The echoed data processing unit 4 performs B-mode processing such as logarithmic compression and envelope detection on echoed data outputted from the transmit/receive beam former 3, creating B-mode data.

The display processing unit 5 performs scan conversion on the B-mode data using a scan converter, creating B-mode image data. The display processing unit 5 then displays a B-mode image on the display unit 6 according to the B-mode image data and displays soft keys, on which an instruction is inputted by an operator, on a display 11 or the like of the operation unit 7, which will be described later.

The display unit 6 is an LCD (Liquid Crystal Display) or an organic EL (Electro-Luminescence) display.

The operation unit 7 includes a keyboard or buttons allowing an operator to input instructions or information and pointing devices such as a trackball.

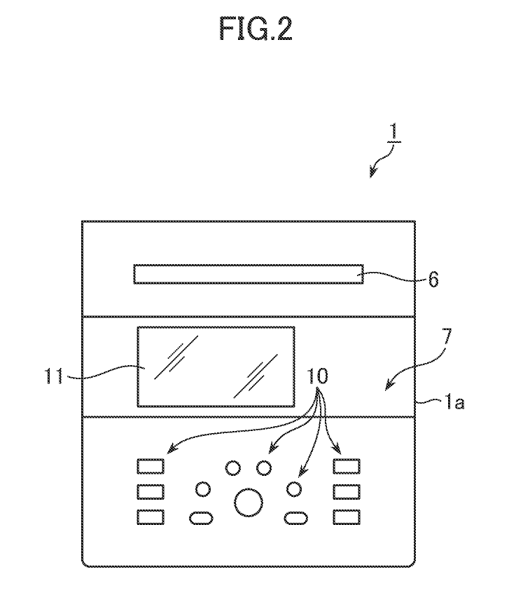

Referring to FIG. 2, the buttons of the operation unit 7 will be described below. FIG. 2 only schematically shows the outline of the operation unit 7. Only the buttons will be described below. The ultrasonic diagnostic device body 1a has hard keys 10 that constitute the buttons. Moreover, the ultrasonic diagnostic device body 1a has the touch-panel display 11. On the display 11, the display processing unit 5 displays the soft keys (not shown in FIG. 2) that constitute the buttons.

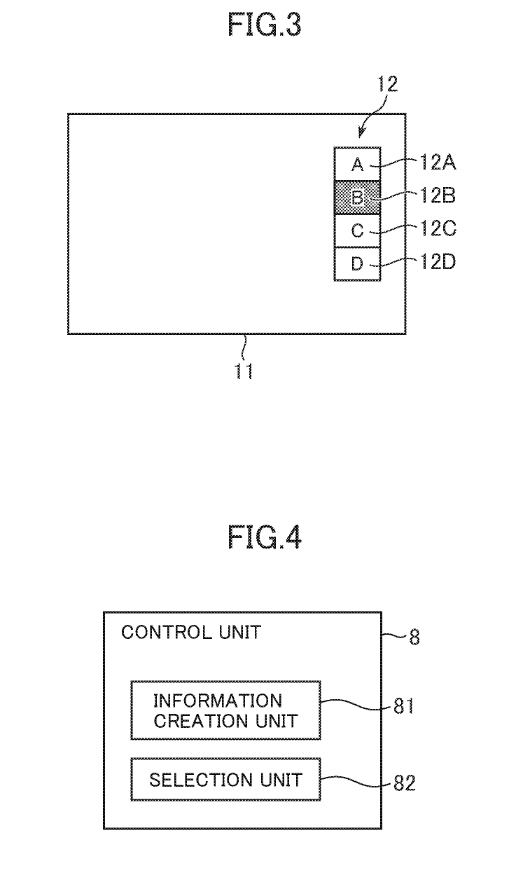

As shown in FIG. 3, a probe selection button 12 is displayed as the soft keys on the display 11. The probe selection button 12 includes four probe selection buttons 12A, 12B, 12C, and 12D. With a press on the probe selection button 12A, the ultrasonic probe 2A is selected. With a press on the probe selection button 12B, the ultrasonic probe 2B is selected. With a press on the probe selection button 12C, the ultrasonic probe 2C is selected. With a press on the probe selection button 12D, the ultrasonic probe 2D is selected. The probe selection button 12 may be pressed by a cursor (not shown) displayed on the display 11 or an operator's touch on the display 11. The selection of the ultrasonic probes 2A, 2B, 2C, and 2D will be described later.

The display processing unit 5 displays the probe selection button 12, which corresponds to the selected ultrasonic probe 2, in a different color from other probe selection buttons. In FIG. 3, the probe selection button 12B is displayed in a different color from the probe selection buttons 12A, 12C, and 12D (In FIG. 3, the probe selection button 12B is indicated by dots).

The control unit 8 is a processor, e.g., a CPU (Central Processing Unit). The control unit 8 reads programs stored in the memory unit 9 and controls the units of the ultrasonic diagnostic device 1. For example, the control unit 8 reads the programs stored in the memory unit 9 and executes the functions of the transmit/receive beam former 3, the echoed data processing unit 4, and the display processing unit 5 according to the read programs.

The control unit 8 may perform only some or all of the functions of the transmit/receive beam former 3, the echoed data processing unit 4, and the display processing unit 5 according to the programs. If the control unit 8 performs only some of the functions, other functions may be performed by hardware such as circuits.

The functions of the transmit/receive beam former 3, the echoed data processing unit 4, and the display processing unit 5 may be realized by hardware such as circuits.

The control unit 8 reads the programs stored in the memory unit 9 and then performs the functions of an information creation unit 81 and a selection unit 82 that are shown in FIG. 4. The information creation unit 81 creates information on the frequency of use of the ultrasonic probe 2 (information creating function). The creation of information will be specifically described later. The information creating function of the information creation unit 81 is an embodiment of the information creating function of the present invention.

The selection unit 82 selects one to be used from the ultrasonic probes 2A, 2B, 2C, and 2D (selecting function). In this case, “selection” means that one of the ultrasonic probes 2 connected to the ultrasonic diagnostic device body 1a via the connectors is electrically connected to the ultrasonic diagnostic device body 1a so as to transmit and receive electrical signals between the ultrasonic diagnostic device body 1a and the ultrasonic probe 2. The ultrasonic probe selected by the selection unit 82 transmits and receives ultrasonic waves.

In response to an input of one of the probe selection buttons 12A to 12D, the selection unit 82 selects the ultrasonic probe. Even if the probe selection buttons 12A to 12D are not pressed, as will be described later, the selection unit 82 selects the ultrasonic probe based on information on the frequency of use of the ultrasonic probes 2. The color of the probe selection button 12 corresponding to the ultrasonic probe 2 selected by the selection unit 82 is displayed in a different color from the other probe selection buttons 12.

The selecting function of the selection unit 82 is an embodiment of the selecting function of the present invention. The selection unit 82 is an embodiment of the selection unit of the present invention.

The memory unit 9 is an HDD (Hard Disk Drive) and/or a semiconductor memory, e.g., RAM (Random Access Memory), and/or a ROM (Read Only Memory). For example, the memory unit 9 stores the log data of the ultrasonic diagnostic device 1 in addition to the programs. The log data is an embodiment of information on a use situation of the ultrasonic diagnostic device according to the present invention.

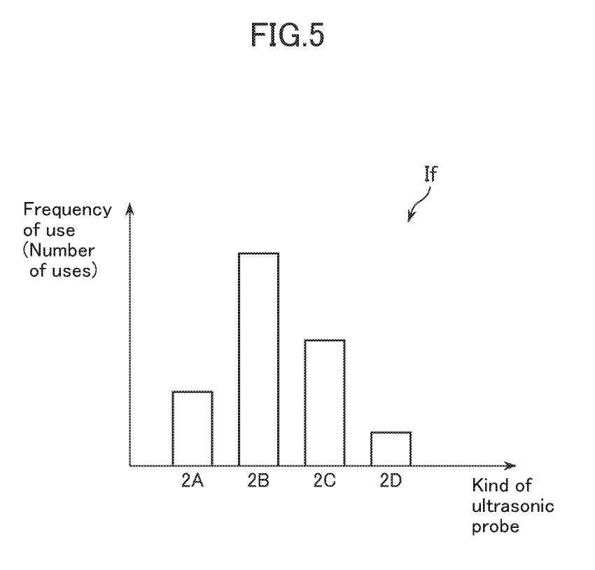

The effect of the ultrasonic diagnostic device 1 of the present example will be described below. The log data of the ultrasonic diagnostic device 1 contains information that specifies the ultrasonic probe 2 selected by the selection unit 82. The log data is stored in the memory unit 9. The information creation unit 81 analyzes the log data of the ultrasonic diagnostic device 1 and creates information on the frequency of use of the ultrasonic probe 2 connected to the ultrasonic diagnostic device body 1a. In the present example, as shown in FIG. 5, information If on the frequency of use of the ultrasonic probes 2A, 2B, 2C, and 2D is created. The created information If on the frequency of use is stored in the memory unit 9. The information creation unit 81 periodically updates the information If on the frequency of use based on the log data. The information creation unit 81 may update the information If on the frequency of use when an instruction for an update is inputted on the operation unit 7.

The selection unit 82 selects the most frequently used ultrasonic probe 2 in the information If on the frequency of use. In this example, the selection unit 82 selects the ultrasonic probe 2B.

The ultrasonic probe 2 is selected based on the information If on the frequency of use, for example, when the operator turns on the ultrasonic diagnostic device 1. Thus, in response to turning-on of the ultrasonic diagnostic device 1 by the operator, the most frequently used ultrasonic probe 2B in the ultrasonic diagnostic device 1 is automatically selected. On the display 11, the probe selection button 12B is displayed in a different color from the probe selection buttons 12A, 12C, and 12D to indicate the selection of the ultrasonic probe 2B (See FIG. 3).

If the automatically selected ultrasonic probe 2B is used, the operator starts a test with the ultrasonic probe 2. If the operator requires the ultrasonic probes 2A, 2C, and 2D other than the ultrasonic probe 2B, the operator presses the probe selection button 12 corresponding to the ultrasonic probe 2 to be used. This selects the ultrasonic probe 2 corresponding to the pressed probe selection button 12.

The ultrasonic diagnostic device 1 of the present example automatically selects the ultrasonic probe 2 most frequently used in the ultrasonic diagnostic device 1. If a test is conducted using the ultrasonic probe 2, the operator does not need to select the ultrasonic probe 2.

The operator may turn on or off the function of selecting the ultrasonic probe 2 based on the information If on the frequency of use by means of the selection unit 82.

Modifications of the first embodiment will be described below, starting from a first modification. In the first modification, the information creation unit 81 analyzes the log data of the ultrasonic diagnostic device 1 and creates information on the frequency of use of the ultrasonic probes 2A, 2B, 2C, and 2D for each subject in the ultrasonic diagnostic device 1.

Before a test is conducted using the ultrasonic diagnostic device 1, the operator inputs subject identification information (including a subject's name and ID) on the operation unit 7. The log data includes information for identifying a subject and information on the ultrasonic probe 2 used for the subject. Thus, analyses on the log data can create information on the frequency of use of the ultrasonic probes 2A, 2B, 2C, and 2D for each subject.

In the first modification, the operator inputs an instruction on the operation unit 7 to select the ultrasonic probe 2 most frequently used for a subject to be tested. Subsequently, when subject identification information is inputted on the operation unit 7, the selection unit 82 selects the most frequently used ultrasonic probe in information on the frequency of use of the ultrasonic probes 2A, 2B, 2C, and 2D for the subject. Thus, in a test using the ultrasonic probe 2 most frequently used for the subject, the ultrasonic probe 2 to be used is selected only by inputting subject identification information. Thus, the operator does not need to select the ultrasonic probe 2.

A second modification will be described below. In the second modification, the information creation unit 81 analyzes the log data of the ultrasonic diagnostic device 1 and creates information on the frequency of use of the ultrasonic probes 2A, 2B, 2C, and 2D for each operator of the ultrasonic diagnostic device 1.

Before a test is conducted using the ultrasonic diagnostic device 1, the operator inputs operator identification information (including a subject's name and ID). The log data includes information for identifying an operator and information on the ultrasonic probe 2 used for the operator. Thus, analyses on the log data can create information on the frequency of use of the ultrasonic probes 2A, 2B, 2C, and 2D for each operator.

In the second modification, the operator inputs an instruction on the operation unit 7 to select the ultrasonic probe 2 most frequently used for the operator of a test. Subsequently, when the operator identification information is inputted on the operation unit 7, the selection unit 82 selects the most frequently used ultrasonic probe in information on the frequency of use of the ultrasonic probes 2A, 2B, 2C, and 2D for the operator. Thus, in a test using the ultrasonic probe 2 most frequently used for the operator, the ultrasonic probe 2 to be used is selected only by inputting operator identification information. Thus, the operator does not need to select the ultrasonic probe 2.

In some hospitals, a specific tester may conduct a test at a specific time on a specific day of a week. Thus, the information creation unit 81 analyzes the log data of the ultrasonic diagnostic device 1 and creates information on the frequency of use of the ultrasonic probes 2A, 2B, 2C, and 2D at each time in the ultrasonic diagnostic device 1.

The time includes a day of a week and a time of day. The time may be, for example, about two or three hours or a certain time period in the morning or evening. The ultrasonic diagnostic device 1 has the time function of specifying a current time, a day of a week, and a date.

The log data includes information on the type of selected ultrasonic probe 2 with information on a day of a week and a time when the ultrasonic probe 2 is selected. Thus, an analysis of the log data can create information on the frequency of use of the ultrasonic probes 2A, 2B, 2C, and 2D.

If information on the frequency of use of the ultrasonic probes 2A, 2B, 2C, and 2D is created at each time, an operator may input an instruction on the operation unit 7 so as to select the most frequently used ultrasonic probe 2 at each time. In this case, for example, when the ultrasonic diagnostic device 1 is turned on, the selection unit 82 selects the most frequently used ultrasonic probe on the day of a week and at the time of the turn-on based on information on the frequency of use of the ultrasonic probes 2A, 2B, 2C, and 2D at each time. Thus, in a test using the ultrasonic probe 2 most frequently used on the day of a week and at the time of the turn-on, the ultrasonic probe 2 to be used is selected only by turning on the ultrasonic diagnostic device 1. Thus, the operator does not need to select the ultrasonic probe 2.

A third modification will be described below. In the third modification, the information creation unit 81 analyzes the log data of the ultrasonic diagnostic device 1 and creates information on the frequency of use of the ultrasonic probes 2A, 2B, 2C, and 2D for each test type in the ultrasonic diagnostic device 1. The test type is, for example, a test part or a test purpose.

Before a test is conducted using the ultrasonic diagnostic device 1, the operator inputs test-type identification information on the operation unit 7. The log data includes information for identifying a test type and information on the ultrasonic probe 2 used for the test type. Thus, analyses on the log data can create information on the frequency of use of the ultrasonic probes 2A, 2B, 2C, and 2D for each test type.

In the third modification, the operator inputs an instruction on the operation unit 7 to select the ultrasonic probe 2 most frequently used for each test type. Subsequently, when the test type identification information is inputted on the operation unit 7, the selection unit 82 selects the most frequently used ultrasonic probe in information on the frequency of use of the ultrasonic probes 2A, 2B, 2C, and 2D for the test type. Thus, in a test using the ultrasonic probe 2 most frequently used for the test type, the ultrasonic probe 2 to be used is selected only by inputting test-type identification information. Thus, the operator does not need to select the ultrasonic probe 2.

Also in the modifications, the operator may turn on or off the function of selecting the ultrasonic probe 2 by means of the selection unit 82 based on the information on the frequency of use of the ultrasonic probes 2A, 2B, 2C, and 2D.

### Second Embodiment

A second embodiment will be described below. The explanation of the same matters as in the first embodiment is omitted.

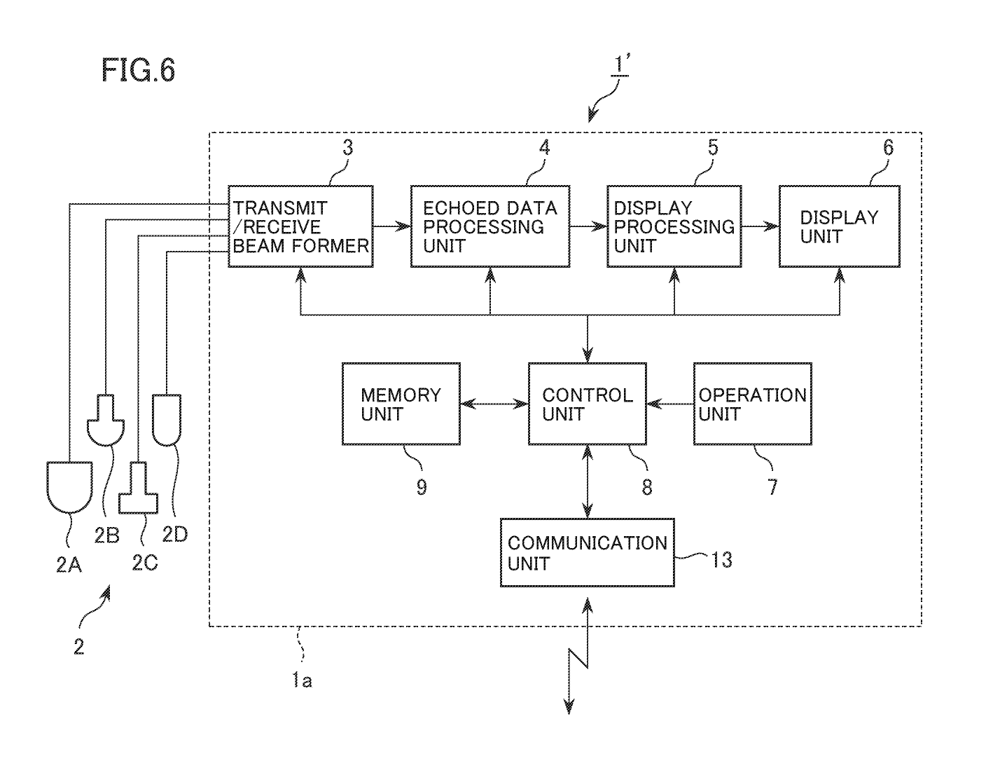

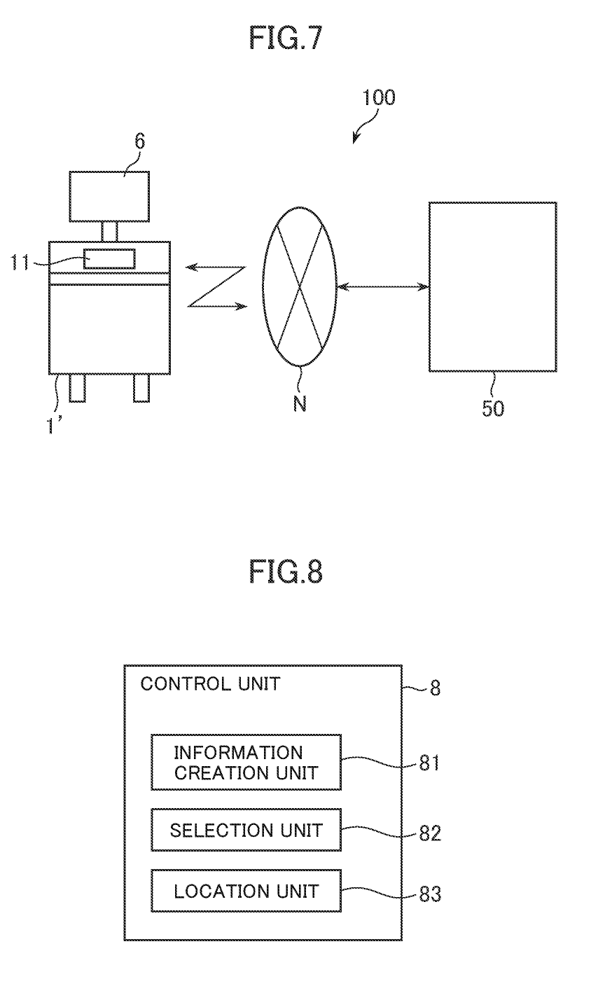

As shown in FIG. 6, ultrasonic diagnostic device 1′ includes a communication unit 13. As shown in FIG. 7, the communication unit 13 is connected to a network N, which constitutes a system 100, via radio communications or the like. The network N is, for example, a hospital LAN (Local Area Network). A server 50 constituting the system 100 is connected to the network N. The server 50 may be installed in a hospital having the network N. The ultrasonic diagnostic device 1′ is connected to the server 50 via the network N.

The server 50 has a known configuration as a computer and the specific configuration thereof is omitted. Ultrasonic image data such as B-mode image data obtained in the ultrasonic diagnostic device 1′ is stored in the server 50. The server 50 is an embodiment of the computer of the present invention.

The server 50 may be installed in a facility other than a hospital. In this case, the ultrasonic diagnostic device 1′ is connected to the server 50 via the network N and a network between the hospital and the facility having the server 50.

As shown in FIG. 8, the control unit 8 of the ultrasonic diagnostic device 1′ includes a location unit 83 in addition to the information creation unit 81 and the selection unit 82. The location unit 83 specifies a position in a facility where the ultrasonic diagnostic device 1′ is used (locating function). In the present example, as will be described later, the location unit 83 specifies an examination room R in a hospital where the ultrasonic diagnostic device 1′ is used. The locating function is an embodiment of the locating function of the present invention. The locating unit 83 is an embodiment of the location unit of the present invention.

In the present example, the information creation unit 81 analyzes the log data of the ultrasonic diagnostic device 1′ and creates information on the frequency of use of the ultrasonic probes 2A, 2B, 2C, and 2D for examination rooms R1 to R8. The information on the frequency of use is an embodiment of the information on the use conditions of the ultrasonic diagnostic device according to the present invention. Moreover, the function of creating information on the frequency of use by means of the information creation unit 81 of the present example is an embodiment of the information creating function of the present invention.

The selection unit 82 selects an ultrasonic probe 2 most frequently used at the use position of the ultrasonic diagnostic device 1′. The selecting function of the selection unit 82 is an embodiment of the selecting function of the present invention and an embodiment of a setting function for a setting of the use conditions of the ultrasonic diagnostic device according to a position specified by the locating function. The selection unit 82 is an embodiment of the selection unit and the setting unit of the present invention. The selection of the ultrasonic probe 2 of the selection unit 82 is an embodiment of the setting of use conditions according to the present invention. The type of ultrasonic probe 2 is an embodiment of the use conditions of the ultrasonic diagnostic device.

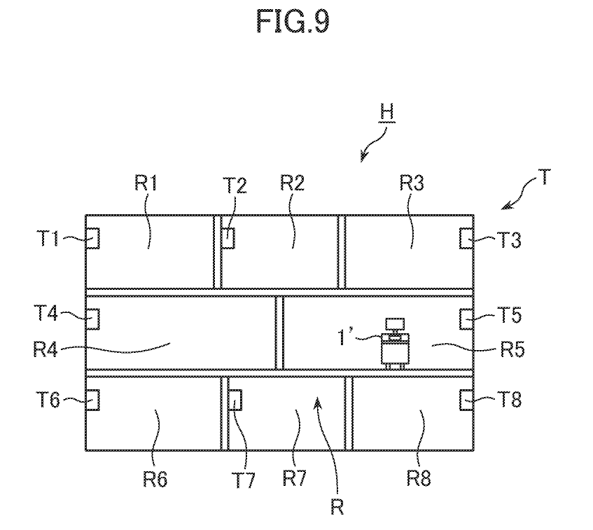

The effect of the present example will be described below. FIG. 9 shows examination rooms R in a hospital H. In the present example, the examination rooms R1 to R8 are provided. The ultrasonic diagnostic device 1′ is used in one of the examination rooms R1 to R8. In FIG. 9, the ultrasonic diagnostic device 1′ is located in the examination room R5. A test type is determined in each of the examination rooms R1 to R8.

Each of the examination rooms R1 to R8 has wireless communications terminals T (T1 to T8) for a wireless LAN. If the ultrasonic diagnostic device 1′ is located in one of the examination rooms R1 to R8, the communication unit 13 wirelessly communicates with the wireless communication terminal T provided in the examination room R having the ultrasonic diagnostic device 1′.

In the present example, as log data, information is provided for specifying the wireless communication terminals T1 to T8 having wirelessly communicated with the communication unit 13. When the wireless communication terminal T that communicates with the communication unit 13 is specified from the wireless communication terminals T1 to T8, the examination room R having the ultrasonic diagnostic device 1′ is specified from the examination rooms R1 to R8. In this case, the log data includes the time of the selection of the ultrasonic probe 2 and the time of the wireless communication of the communication unit 13. Thus, the information creation unit 81 analyzes the log data, specifies the wireless communication terminal T having communicated with the communication unit 13 and the ultrasonic probe 2 used at the time of the wireless communication, and creates information on the frequency of use of the ultrasonic probes 2A, 2B, 2C, and 2D in the examination rooms R1 to R8. The examination rooms R1 to R8 are specified by the location unit 83 based on the wireless communication terminal T having communicated with the communication unit 13.

In the second embodiment, the operator inputs an instruction on the operation unit 7 to select the ultrasonic probe 2 most frequently used in the examination room having the ultrasonic diagnostic device 1′. Subsequently, when the ultrasonic diagnostic device 1′ is located in one of the examination rooms R1 to R8, the selection unit 82 selects the most frequently used ultrasonic probe 2 in the examination room R having the ultrasonic diagnostic device 1′.

The selection of the ultrasonic probe 2 by the selection unit 82 will be more specifically described below. First, when the communication unit 13 starts wireless communications with the wireless communication terminal T, the examination room R having the ultrasonic diagnostic device 1′ is specified from the examination rooms R1 to R8. Subsequently, based on the information on the frequency of use of the ultrasonic probes 2A, 2B, 2C, and 2D in the specified examination room R, the selection unit 82 specifies the ultrasonic probe 2 most frequently used in the examination room R and then selects the ultrasonic probe 2.

According to the present example, when the communication unit 13 starts wireless communications with the wireless communication terminal T, the selection unit 82 selects the ultrasonic probe 2 most frequently used in the examination room R having the wireless communication terminal T. Hence, in a test with the most frequently used ultrasonic probe 2, the operator does not need to select the ultrasonic probe 2.

Also in the present example, the operator may turn on or off the function of selecting the ultrasonic probe 2 by means of the selection unit 82 based on the information on the frequency of use of the ultrasonic probes 2A, 2B, 2C, and 2D.

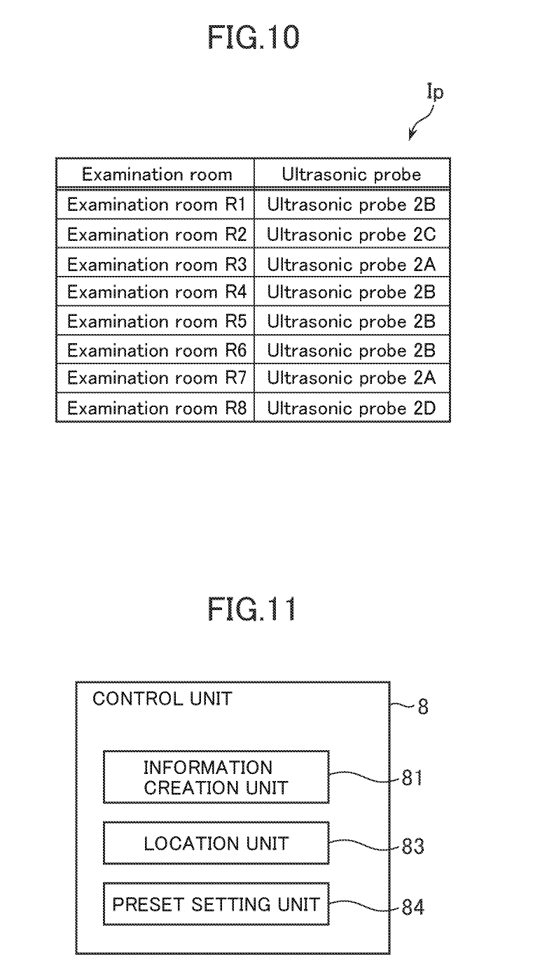

Modifications of the second embodiment will be described below, starting from a first modification. As shown in FIG. 10, the information creation unit 81 creates type information Ip on the ultrasonic probes 2A, 2B, 2C, and 2D used for the examination rooms R1 to R8. In the information Ip, the ultrasonic probe 2 determined for each of the examination rooms R1 to R8 is the most frequently used ultrasonic probe 2 in each of the examination rooms R1 to R8. The information Ip is an embodiment of information on the use conditions of the ultrasonic diagnostic device according to the present invention.

The operator inputs information on types of ultrasonic probes 2 used in the examination rooms R1 to R8, by means of the operation unit 7. The information creation unit 81 creates the information Ip based on the input on the operation unit 7. The input of the type information on the ultrasonic probes 2 is an embodiment of an input of information on the use conditions of the ultrasonic diagnostic device according to the present invention.

The selection unit 82 specifies, based on the information Ip, the ultrasonic probe 2 used in the examination room R specified by the location unit 83 and then selects the ultrasonic probe 2.

A second modification will be described below. As shown in FIG. 11, the control unit 8 includes a preset setting unit 84, which replaces the selection unit 82, in addition to the information creating unit 81 and the location unit 83.

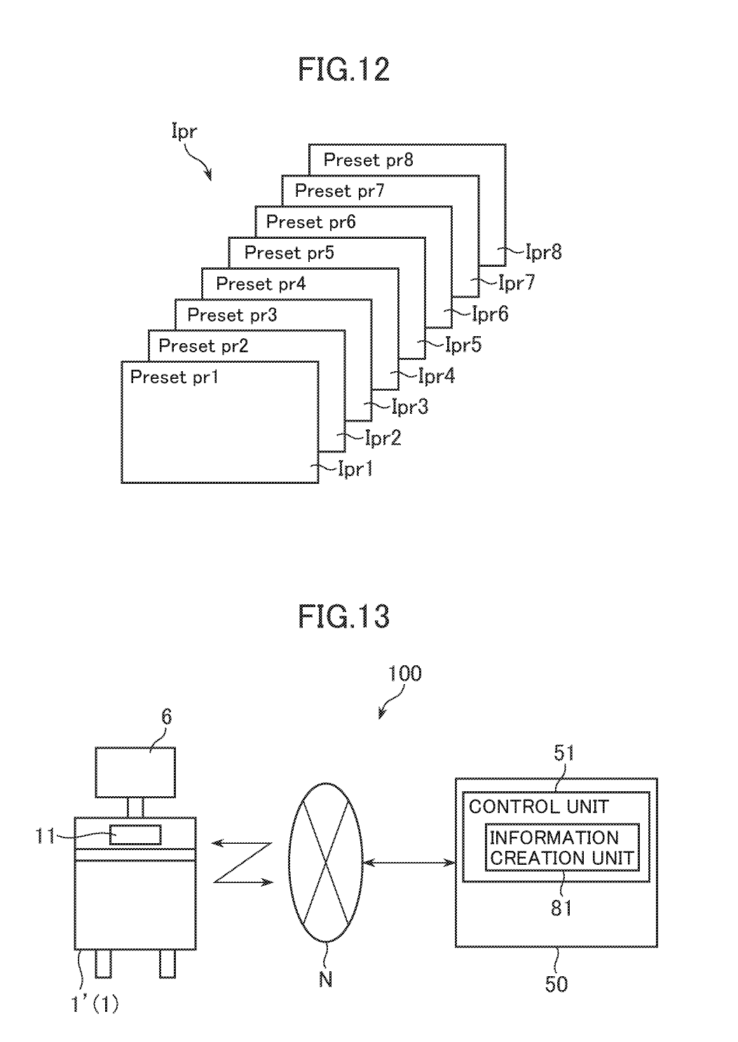

The information creation unit 81 analyzes the log data of the ultrasonic diagnostic device 1′ and creates preset information Ipr set for the ultrasonic diagnostic device 1′. As shown in FIG. 12, the preset information Ipr is created for the examination rooms R1 to R8 (preset information Ipr1 to Ipr8). In FIG. 12, the presets pr1 to pr8 indicate presets that are set for the examination rooms R1 to R8, respectively. The presets pr1 to pr8 are an embodiment of the use conditions of the ultrasonic diagnostic device according to the present invention.

The presets pr1 to pr8 are use conditions that are set for the ultrasonic diagnostic device 1′ according to the test type when the ultrasonic diagnostic device 1′ is used. Since test types are determined for the examination rooms R1 to R8, the presets are determined for the examination rooms R1 to R8. For example, the presets include a measurement button or a body pattern on a display 11 of the operation unit 7, ultrasonic transmission/reception parameters, and functions allocated to hard keys 10 of the operation unit 7.

The preset setting unit 84 specifies, based on the preset information Ipr, the preset that is set for the ultrasonic diagnostic device 1′ in the examination room R located by the location unit 83, and then the preset setting unit 84 sets the preset.

In the second modification, an operator may input preset information for the respective examination rooms R1 to R8 by means of the operation unit 7. In this case, the information creation unit 81 creates the preset information Ipr based on the input of the operation unit 7.

According to the second modification, when the communication unit 13 starts wireless communications with the wireless communication terminal T, the preset set for the examination room R having the wireless communication terminal T is set for the ultrasonic diagnostic device 1′. Thus, the operator does not need to set the preset.

The operator may turn on or off the function of setting the preset for the ultrasonic diagnostic device 1′ based on the preset information Ipr by means of the preset setting unit 84.

The present invention was described in accordance with the embodiments. As a matter of course, the present invention may be changed in various ways without changing the scope of the invention. For example, as shown in FIG. 13, the control unit 51 of the server 50 may include the information creation unit 81 in the system 100. Specifically, the control unit 61 is a processor, e.g., a CPU. The server 50 is an embodiment of a computer in the present invention.

If the control unit 51 of the server 50 has the information creation unit 81, the control unit 8 may or may not have the information creation unit 81.

If the server 50 has the information creation unit 81, the information creation unit 81 creates the information based on the log data of the ultrasonic diagnostic device 1′, the log data being inputted to the server 50 through the network. Subsequently, the information created by the information creation unit 81 is inputted from the server 50 to the ultrasonic diagnostic device 1′ through the network N. The selection unit 82 and the preset setting unit 84 select the ultrasonic probe 2 or set the preset based on the information inputted from the server 50.

The information may be created by the information creation unit 81 of the server 50 based on the input of information on the input unit of the server 50 or the operation unit 7 of the ultrasonic diagnostic device 1′.

The ultrasonic diagnostic device 1 may be connected to the network N. Also in this case, the information creation unit 81 may be provided in the server 50.

The display unit 6 may display an ultrasonic image other than a B-mode image. In this case, the echoed data processing unit 4 performs necessary processing on echoed data outputted from the transmit/receive beam former 3. Subsequently, the display processing unit 5 performs scan conversion on the obtained data to create image data, and then an ultrasonic image based on the image data is displayed on the display unit 6.

1. An ultrasonic diagnostic device comprising:
a plurality of ultrasonic probes; and
a processor that performs a selecting function according to a program so as to select a most frequently used ultrasonic probe based on information on frequency of use of the ultrasonic probes, the information being specified by information on a use situation of the ultrasonic diagnostic device.

2. The ultrasonic diagnostic device according to claim 1, further comprising a processor that performs an information creating function according to a program so as to create information on frequency of use of the ultrasonic probes based on information on a use situation of the ultrasonic diagnostic device.

3. The ultrasonic diagnostic device according to claim 1, wherein the selecting function selects the most frequently used ultrasonic probe in the ultrasonic diagnostic device.

4. The ultrasonic diagnostic device according to claim 1, further comprising an input unit that allows an operator to input subject identification information,
wherein the information on the frequency of use of the ultrasonic probes is specified for each subject, and
the selecting function selects the most frequently used ultrasonic probe for a subject inputted on the input unit.

5. The ultrasonic diagnostic device according to claim 1, further comprising an input unit that allows an operator to perform an input for identification of the operator,
wherein the information on the frequency of use of the ultrasonic probes is specified for each operator, and
the selecting function selects the most frequently used ultrasonic probe for the operator inputted on the input unit.

6. The ultrasonic diagnostic device according to claim 1, further comprising an input unit that allows an operator to perform an input for identification of a test type,
wherein the information on the frequency of use of the ultrasonic probes is specified for each test type, and
the selecting function selects the most frequently used ultrasonic probe for the test type inputted on the input unit.

7. The ultrasonic diagnostic device according to claim 1, wherein the information on the frequency of use of the ultrasonic probes is specified at each time, and
the selecting function selects the most frequently used ultrasonic probe at a time when the ultrasonic diagnostic device is used.

8. The ultrasonic diagnostic device according to claim 1, wherein the information on the frequency of use of the ultrasonic probes is specified at each use position of the ultrasonic diagnostic device, and
the selecting function selects the most frequently used ultrasonic probe at a use position of the ultrasonic diagnostic device.

9. An ultrasonic diagnostic device comprising:
a plurality of ultrasonic probes; and
a selection unit that selects a most frequently used ultrasonic probe based on information on frequency of use of the ultrasonic probes, the information being specified by information on a use situation of the ultrasonic diagnostic device.

10. A system comprising the ultrasonic diagnostic device according to claim 1, and a computer connected to the ultrasonic diagnostic device via a network,
the computer including a processor that performs an information creating function according to a program so as to create information on frequency of use of the ultrasonic probes based on information on a use situation of the ultrasonic diagnostic device.

11. An ultrasonic diagnostic device comprising a processor that performs, according to a program, a locating function that locates the ultrasonic diagnostic device and a setting function that sets a use condition of the ultrasonic diagnostic device according to a position specified by the locating function.

12. The ultrasonic diagnostic device according to claim 11, wherein the processor further performs an information creating function that creates information on the use condition of the ultrasonic diagnostic device according to a use position of the ultrasonic diagnostic device, based on information on the use situation of the ultrasonic diagnostic device and information associated with the use position of the ultrasonic diagnostic device, and
the setting function sets the use condition based on the information on the use condition of the ultrasonic diagnostic device.

13. The ultrasonic diagnostic device according to claim 11, further comprising an input unit for input of information on the use condition of the ultrasonic diagnostic device according to a use position of the ultrasonic diagnostic device,
wherein the setting function sets the use condition based on the information on the use condition of the ultrasonic diagnostic device.

14. The ultrasonic diagnostic device according to claim 11, wherein the use condition of the ultrasonic diagnostic device is the kind of ultrasonic probe.

15. The ultrasonic diagnostic device according to claim 11, wherein the use condition of the ultrasonic diagnostic device is a preset that is set for the ultrasonic diagnostic device according to the use position of the ultrasonic diagnostic device.

16. The ultrasonic diagnostic device according to claim 11, wherein the position of the ultrasonic diagnostic device is a position in a facility where the ultrasonic diagnostic device is used, and a test type at the position in the facility is specified.

17. The ultrasonic diagnostic device according to claim 1 wherein information on the use situation of the ultrasonic diagnostic device is log data.

18. An ultrasonic diagnostic device comprising:
a location unit that locates the ultrasonic diagnostic device; and
a setting unit that sets a use condition of the ultrasonic diagnostic device according to a position specified by the location unit.

19. A system comprising the ultrasonic diagnostic device according to claim 11, and a computer connected to the ultrasonic diagnostic device via a network,
the computer including a processor that performs an information creating function according to a program so as to create information on the use condition of the ultrasonic diagnostic device according to the use position of the ultrasonic diagnostic device.
